(19) United States
(12) Patent Application Publication
(10) Pub. No.: US 2007/0148750 A1
(43) Pub. Date: Jun. 28, 2007

(54) Method for producing monosaccharides from biomass and monosaccharide production device

(75) Inventors: Cyuichi Hoshino, Yokohama-shi (JP); Tomiaki Yamada, Tokyo (JP); Daisuke Taneda, Hitachinaka-shi (JP); Yasuhisa Nagata, Tokyo (JP); Tomoaki Fujii, Fujisawa-shi (JP); Takao Mase, Yokohama-shi (JP); Yoshiki Ueno, Mito-shi (JP)

Correspondence Address: DARBY & DARBY P.C., P. O. BOX 5257, NEW YORK, NY, 10150-5257, US

(73) Assignee: JGC CORPORATION, Tokyo (JP)

(21) Appl. No.: 10/597,962

(22) PCT Filed: Feb. 8, 2005
(86) PCT No.: PCT/JP05/01843

§ 371 Date: Aug. 14, 2006

(30) Foreign Application Priority Data
Feb. 17, 2004 (JP) 2004-039651

(51) Int. Cl. C12P 7/06; C07H 1/00

(52) U.S. Cl. 435/161; 536/124

(57) ABSTRACT

This monosaccharide production method is a method for producing monosaccharides from biomass having a first step 3 in which a raw material biomass is pretreated in 65 to 85 (w/w) % sulfuric acid at a temperature of 30 to 70° C., and a second step 4 in which the first step treatment product pretreated in the first step is subjected to saccharification treatment in 20 to 60 (w/w)% sulfuric acid at a temperature of 40 to 100° C.

### TECHNICAL FIELD

The present invention relates to a biomass conversion technology for effectively utilizing a biomass resource as an energy source or as a raw material for producing various chemical products, and more particularly, to a method for producing monosaccharides from biomass using sulfuric acid, and a monosaccharide production device.

The present application claims priority on Japanese Patent Application No. 2004-39651, filed on Feb. 17, 2004, the content of which is incorporated herein by reference.

### BACKGROUND ART

Research and development has been carried out in the past on technologies for producing monosaccharides such as glucose, xylose and mannose serving as raw materials for the production of ethanol, amino acids, organic acids and various other chemical products from wood-based biomass from coniferous trees, deciduous trees, thinning lumber, processing waste materials or construction waste materials and so on, and from rice straw, bagasse, beet pulp and various other types of herbaceous plants.

In particular, the “Arkenol process” proposed by the US Arkenol, Inc. is known as a method for hydrolyzing biomass using sulfuric acid (see, for example, Japanese Unexamined International Patent Publication No. H11-506934).

In this “Arkenol process”, a two-stage hydrolysis method is employed in which the cellulose and hemicellulose contained in a biomass are each reacted separately to efficiently convert to monosaccharides. The flow chart of this production method is shown in FIG. 4. In the “Arkenol process”, the decrystallization (1) and hydrolysis reaction (1) of the first stage are first carried out under mild conditions for the purpose of minimizing degradation of xylose converted from hemicellulose. Next, filtration (1) is carried out, since a large amount of unreacted cellulose is contained in the resulting solid (filter cake) following this filtration (1), a second-stage decrystallization (2) and hydrolysis reaction (2) are carried out on this solid for the purpose of converting the large amount of remaining cellulose to glucose. As a result, the “Arkenol process” is characterized by making it possible to expect overall improvement in the yields of C5- and C6-monosaccharides from hemicellulose and cellulose.

### DISCLOSURE OF THE INVENTION

One of the reasons for carrying out a two-stage hydrolysis reaction in the “Arkenol process” as claimed in the aforementioned Japanese Unexamined International Patent Publication No. H11-506934 is to prevent degradation of monosaccharides (and particularly, xylose) originating in hemicellulose. When the inventors of the present invention conducted tests to confirm degradation of hemicellulose, remarkable degradation of xylose was unexpected not confirmed in the first stage of the hydrolysis reaction (1).

This means that it is not necessary to intentionally carry out the first stage hydrolysis reaction (1) under mild conditions.

In addition, in the “Arkenol process”, temperature, sulfuric acid concentration and other conditions in the first and second stage hydrolysis reactions (1) and (2) are adjusted to be the same. With respect to this, the inventors of the present invention attempted the second stage hydrolysis reaction (2) by adding sulfuric acid to the residue resulting after the first stage hydrolysis reaction (1) for the purpose of improving the final monosaccharide conversion rate.

However, contrary to expectations, the concentration of the sugar that formed following the second stage hydrolysis reaction (2) was extremely low.

When filtrates (1) and (2) following two hydrolysis reactions (1) and (2) are mixed in a two-stage hydrolysis reaction method, there was the problem of the overall concentration of the sugar solution decreasing, as well as the problem of increased device costs due to requiring two filtration steps.

In consideration of the aforementioned problems of the prior art, an object of the present invention is to provide a method for producing monosaccharides by a simplified process when producing monosaccharides from biomass.

In addition, an object of the present invention is to provide a monosaccharide production device having a reduced equipment scale and costs.

A first aspect of the present invention is a method for producing monosaccharides from biomass comprising: a first step in which a raw material biomass is pretreated in 65 to 85 (w/w) (w/w) % sulfuric acid at a temperature of 30 to 70° C., and a second step in which the first step treatment product pretreated in the first step is subjected to saccharification treatment in 20 to 60 (w/w) (w/w) % sulfuric acid at a temperature of 40 to 100° C.

The aforementioned monosaccharide production method may further have a third step in which the second step treatment product resulting from saccharification treatment in the second step is subjected to monosaccharification treatment in 0.5 to 5 (w/w) (w/w)% sulfuric acid at a temperature of 110 to 150° C.

The aforementioned monosaccharide production method may further have a step 2A, in which the treatment product of the second step resulting from saccharification treatment in the second step is subjected to filtration, and a step 2B in which the filtrate following step 2A is separated into sugar and acid.

The first step may also have a step in which the sulfuric acid is sprayed onto and mixed with the biomass followed by kneading.

In the aforementioned monosaccharide production method, the weight-based mixing ratio of the sulfuric acid to biomass is preferably 0.3 to 5.0.

In the second step, a washing filtrate, obtained by washing the solid after step 2A, may be used.

In the aforementioned monosaccharide production method, a simulated moving bed chromatographic separation device may be used for the separation into sugar and acid in step 2B.

In the aforementioned monosaccharide production method, low-concentration sulfuric acid after step 2B may be used for the sulfuric acid of the second step.

In the aforementioned monosaccharide production method, the biomass is a cellulose-based biomass.

A second aspect of the present invention is a monosaccharide production device provided with: a sulfuric acid spraying and mixing device, which sprays 65 to 85 (w/w) % sulfuric acid onto a raw material biomass and mixes the sulfuric acid and biomass by rotating to obtain a sulfuric acid-sprayed/mixed biomass, a continuous kneading device which kneads the sulfuric acid-sprayed/mixed biomass from the sulfuric acid spraying and mixing device by applying shear force to obtain a kneaded product, and a hydrolysis reaction device which adds water or low-concentration sulfuric acid to the first step treatment product in the form of the kneaded product from the continuous kneading device to dilute the sulfuric acid concentration to 20 to 60 (w/w) % followed by treatment at a temperature of 40 to 100° C.; wherein, sequential intermediate products are continuously sent from the sulfuric acid-spraying/mixing device to the hydrolysis reaction device.

According to the method for producing monosaccharides from biomass of the present invention, the process can be simplified by carrying out a single saccharification treatment by a hydrolysis reaction. and the monosaccharide conversion rate can be improved.

### BRIEF DESCRIPTION OF THE DRAWINGS

FIG. 1 is a flow chart of a monosaccharide production method as claimed in a first embodiment of the present invention;

FIG. 2 is a flow chart of a monosaccharide production method as claimed in a second embodiment of the present invention;

FIG. 3 is a schematic drawing of a monosaccharide production device in which spraying/mixing, kneading and hydrolysis are carried out continuously as claimed in an embodiment of the present invention; and

FIG. 4 is a flow chart of a monosaccharide production method as claimed in the Arkenol process.

### BEST MODE FOR CARRYING OUT THE INVENTION

The following provides a detailed explanation of examples of the monosaccharide production method as claimed in the embodiments of the present invention.

### First Embodiment

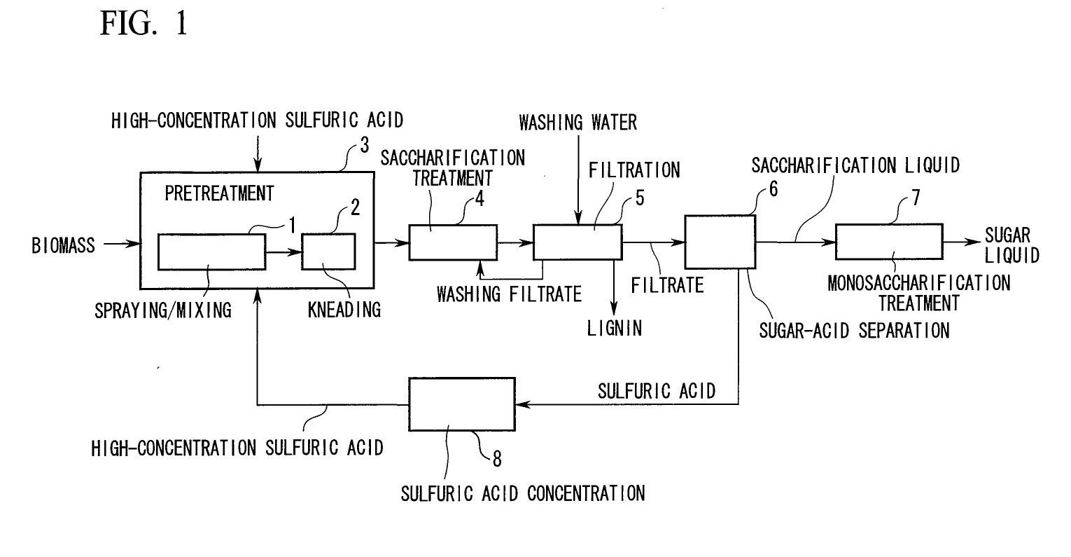

FIG. 1 is a flow chart of a monosaccharide production method as claimed in a first embodiment of the present invention. This monosaccharide production method of the present invention is composed of a first step 3 for carrying out pretreatment for decrystallizing and solubilizing a cellulose and hemicellulose contained in biomass, and a second step 4 for carrying out saccharification treatment for forming monosaccharides by a hydrolysis reaction.

Moreover, in the present embodiment, a step 2A 5, for carrying out filtration on the treatment product of the second step, is present after the second step 4, a step 2B 6, for separating the filtrate into sugar and acid, is present after this step 2A 5, and a third step 7, for carrying out monosaccharification treatment for converting unreacted oligosaccharide remaining in the saccharification liquid to monosaccharides, is present after step 2B 6.

Examples of materials used for the raw material biomass include paper, wood, construction materials, grasses, straw, natural fibers and foods. In addition, waste products such as wastepaper, waste wood, waste construction materials, leftover food and other industrial waste can also be used. Cellulose-based biomass is particularly preferable. Examples of such cellulose-based biomass include biomass having for a main component thereof cellulose, hemicellulose or lignin.

This biomass is preferably in the form of a powder of chips of a suitable size by cutting or crushing, and may be removed of foreign substances as necessary. This biomass is more preferably in the form of rods or sheets having a thickness of 10 mm or less to facilitate a kneading operation to be described later.

(First Step)

In a first step 3 of the present embodiment, the intermolecular bonds of holocellulose (generic term for cellulose and hemicellulose) of biomass are dissociated in sulfuric acid having a concentration 65 to 85 (w/w)%, and preferably 70 to 75 (w/w)%, at a temperature of 30 to 70° C., and preferably 40 to 55° C., to carry out decrystallization and solubilization treatment. As a result of this first step 3, saccharification treatment proceeds easily by the hydrolysis reaction of cellulose or hemicellulose in the second step 4.

At this time, the reason for making the concentration of sulfuric acid 65 to 85 (w/w)% is that, if the concentration of sulfuric acid is less than 65 (w/w)%, the cellulose decrystallization and solubilization rate decreases, while if the concentration of sulfuric acid exceeds 85 (w/w)%, degradation of the solubilized oligosaccharides and monosaccharides is promoted, while also resulting in the problem of requiring a large amount of energy in a sulfuric acid recovery and concentration step 8.

In addition, the reason for making the treatment temperature 30 to 70° C. is that, since the decrystallization and solubilization reaction generates heat, if the treatment temperature exceeds 80 to 90° C., the temperature rises rapidly causing the reaction to proceed out of control and leading to a decrease in the yield of monosaccharide. Moreover, the treatment time of first step 3 is preferably 0.5 to 30 minutes.

In addition, highly concentrated sulfuric acid following the sulfuric acid concentration step 8 to be described later can be used for the sulfuric acid having a concentration of 65 to 85 (w/w)% used here.

In the first step 3, the amount of sulfuric acid (converted at 100 (w/w)% relative to the weight of the biomass (absolute dry weight) is preferably such that the weight ratio of sulfuric acid to biomass is 0.3 to 5.0. As a result of making the weight ratio of sulfuric acid to biomass 0.3 to 5.0, holocellulose can be decrystallized and solubilized with a smaller amount of sulfuric acid than in the conventional “Arkenol process”, while also enabling the amount of sulfuric used in the entire process to be further reduced.

(Second Step)

The treatment product of the first step, in the form of a highly viscous reaction product that has gone through the first step 3, is sent to a second step 4. In this second step 4, water or low-concentration sulfuric acid is added to the first step treatment product to dilute the concentration of sulfuric acid to 20 to 60 (w/w) %, and preferably 20 to 40 (w/w) %, followed by carrying out saccharification treatment by a hydrolysis reaction at a temperature of 40 to 100° C., and preferably 80 to 100° C.

Since the temperature of the solution rises due to an exothermic reaction when the concentration of sulfuric acid is diluted by adding water or low-concentration sulfuric acid, the amount of energy introduced can be reduced. The treatment time of the second step 4 is preferably 10 to 60 minutes. As a result of this hydrolysis reaction, the solubilized oligosaccarides (cellulose and hemicellulose) are converted to the monosaccarides such as glucose or xylose, and a second step treatment product (slurry) is obtained containing sugar and sulfuric acid.

At this time, the reason for making the sulfuric acid concentration 20 to 60 (w/w)% is that if the concentration of sulfuric acid exceeds 60 (w/w)%, degradation of the oligosaccharides and monosaccharides formed is promoted, resulting in a decrease in the yield of monosaccharide.

In addition, the reason for making the treatment temperature 40 to 100° C. is that if the temperature exceeds 100° C., degradation of the oligosaccharides and monosaccharides formed is similarly promoted, again resulting in a decrease in the yield of monosaccharide.

In addition, a washing filtrate resulting from washing the a solid following step 2A 5 to be described later can be used for the water added to dilute the concentration of sulfuric acid.

(Step 2A)

In the present embodiment, the treatment product of the second step (slurry) containing sugar and sulfuric acid is sent to step 2A 5. In this step 2A 5, the second step treatment product is subjected to filtration, and separated into a filtrate and a solid composed of lignin (filter cake). This filtrate is sent to step 2B 6 to be described later.

In addition, since residual sugar and sulfuric acid are adhered to the solid, the solid is washed from the viewpoint of improving the recovery rates of sugar and sulfuric acid and using the solid lignin as a boiler fuel. Hot water at 50 to 90° C. is used for washing. The washing filtrate resulting from washing the solid is temporarily stored in a separate container.

Next, the solid is washed again using the washing filtrate stored in the separate container. This washing procedure is referred to as the “counter-flow system”, is repeated three to five times, and the final washing filtrate is used as water for diluting the sulfuric acid concentration in the second step 4 as previously described.

This washing filtrate has low concentrations of both sugar and sulfuric acid. Thus, if mixed with the filtrate following step 2A 5, the filtrate is diluted, and the concentrations of the saccharification liquid and sulfuric acid of step 2B 6 decrease resulting in the shortcoming of requiring excessive energy for monosaccharide and sulfuric acid concentration.

However, if this washing filtrate is used in the second step 4, although there is a slight amount of sugar degradation, the sugar and sulfuric acid in the washing filtrate can be used effectively without waste, thereby making it possible to improve the sugar and sulfuric acid recovery rates.

(Step 2B)

The filtrate obtained in step 2A 5 is sent to step 2B 6 where it is separated into sugar and acid. An ordinary chromatographic separation device or ion exchange membrane separation device and so forth can be used for this sugar-acid separation. In particular, a simulated moving bed chromatographic separation device is used preferably.

As described in Japanese Patent Application No. 2003-279997, this simulated moving bed chromatographic separation device has a plurality of columns C1, C2, . . . C8 packed with a filler such as an anion exchange resin connected in series in a closed circuit in the form of a conduit. The filtrate is injected into column C1 in the first stage of this simulated moving bed chromatographic separation device, the effluent consisting mainly of sugar having a fast transit speed (referred to as “raffinate”) is eluted from the second stage column C2, the effluent consisting mainly of sulfuric acid having a slow transit speed (referred to as “extract”) is eluted from the sixth stage column C6 by injection of eluent water, and the raffinate (main component: saccharification liquid) and extract (main component: sulfuric acid) are separated based on the difference in their transit speeds.

At this time, the effluent consisting mainly of sulfuric acid (extract) is sent to a sulfuric acid concentration step 8 to be described later. On the other hand, the effluent consisting mainly of sugar (raffinate) is sent to the third step 7.

(Third Step)

In this third step 7, monosaccharification treatment is carried out for converting the unreacted oligosaccharides remaining in the saccharification liquid following step 2B 6 into monosaccharides. The saccharification liquid (raffinate) following step 2B 6 contains an extremely small amount of sulfuric acid in addition to sugar. This saccharification liquid (raffinate), either used at the current sulfuric acid concentration or after adjusting the concentration thereof, is heated and subjected to a monosaccharification reaction by a hydrolysis reaction. The sulfuric acid concentration at this time is 0.5 to 5 (w/w)% by weight, and preferably 1 to 3 (w/w)% by weight, while the temperature is 110 to 150° C., and preferably 120 to 135° C. In addition, the treatment time is preferably 30 to 90 minutes.

This third step 7 is not present in the “Arkenol process” of the prior art. As a result of carrying out the monosaccharification reaction in the third step 7 following the second step 4, unreacted oligosaccharides remaining in the saccharification liquid (raffinate) are again subjected to hydrolysis, thereby making it possible to further improve the final monosaccharide conversion rate.

(Sulfuric Acid Concentration Step)

The effluent consisting mainly of sulfuric acid (extract) is sent to a sulfuric acid recovery and concentration step 8. multiple effect evaporator for conserving energy can be used for this sulfuric acid concentration. As a result, highly concentrated sulfuric acid concentrated to about 70 to 80 (w/w) % can be used for the sulfuric acid fed to the first step 3 as previously described.

### Second Embodiment

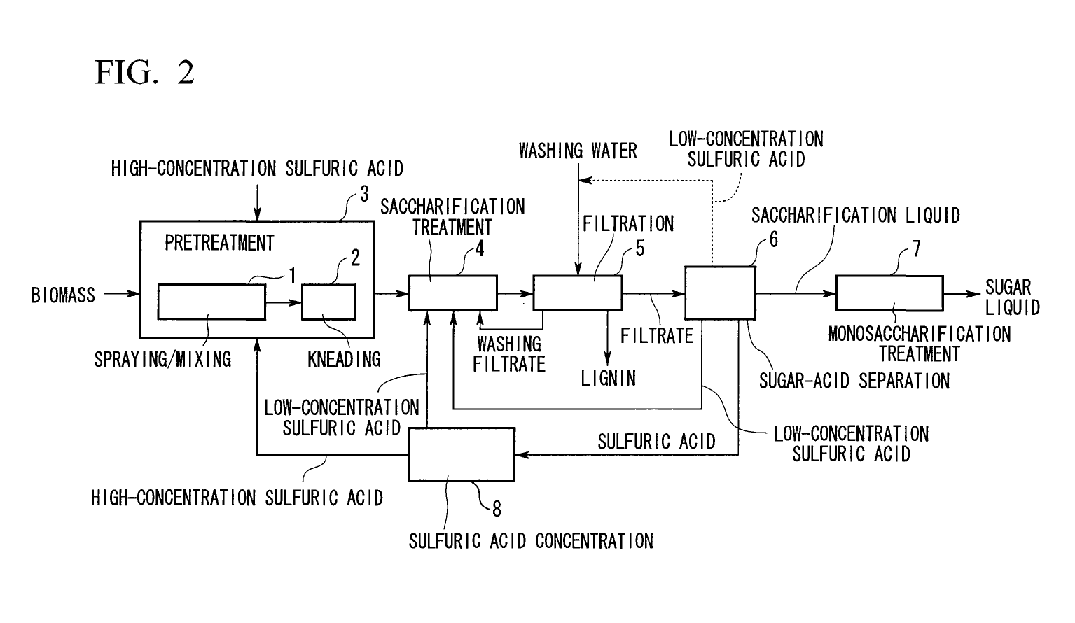

FIG. 2 is a flow chart of a method for producing monosaccharides as claimed in a second embodiment of the present invention. The sulfuric acid recovery and utilization step has been improved in the present embodiment. An explanation is only provided for those portions of the present embodiment which differ from the first embodiment, while an explanation of other portions is omitted since they are the same as the first embodiment.

The sulfuric acid fractioned in step 2B 6 is divided into a high-concentration sulfuric acid fraction (high extract) component and a low-concentration sulfuric acid fraction (low extract) component. In the present embodiment, the resulting low-concentration sulfuric acid fraction (low extract) component is used directly as sulfuric acid for diluting the concentration of the sulfuric acid that is returned to the second step 4. Alternatively, it may also be used instead of the washing water used to wash the solid in step 2A 5.

In addition, the resulting high-concentration sulfuric acid fraction (high extract) component is sent to sulfuric acid concentration step 8. In the sulfuric acid concentration step 8 of the present embodiment, the sulfuric acid is concentrated to the concentration of the second stage.

After having been concentrated to about 30 to 50 (w/w)%, the low-concentration sulfuric acid is returned directly to the second step 4 or returned after having been mixed with a washing filtrate, and is used as sulfuric acid for diluting the concentration of the sulfuric acid of second step 4.

On the other hand, after having been concentrated to about 70 to 80 (w/w)%, the high-concentration sulfuric acid is used as sulfuric acid fed to the first step 3.

Differing from the first embodiment, as a result of returning low-concentration sulfuric acid (low extract) and low-concentration sulfuric acid to the second step 4, the amount of energy required to recover and concentrate the sulfuric acid can be reduced.

In addition, since the addition of sulfuric acid to the second step 4 is not taken into consideration in the first embodiment, the weight-based mixing ratios of sulfuric acid to biomass in the first step 3 and second step 4 are preferably both at the same mixing ratios from the viewpoint of sugar recovery. In the present embodiment, however, since sulfuric acid is added to the second step 4, even if the weight-based mixing ratio of sulfuric acid to biomass in the previous first step 3 is low, that value can be adjusted to be higher in the second step 4, thereby making it possible to ultimately achieve a sugar yield that is equivalent to that of the first embodiment.

Since the amount of sulfuric acid fed to the first step 3 can be reduced, the amount of energy required to recover the sulfuric acid can also be reduced.

Moreover, since sulfuric acid can be added to the second step 4, excessively high viscosity of the second step treatment product (slurry) following the hydrolysis reaction can be prevented, thereby slurry handling in subsequent steps while also making it possible to avoid being unable to obtain a filtrate during filtration in step 2A 5.

In the present invention, in addition to being able to carry out the first step 3 and the second step 4 in the form of batch treatment, the first step 3 can be composed of a step 1 for spraying and mixing sulfuric acid into the biomass, and a step 2 for kneading this biomass sprayed and mixed with sulfuric acid. Sugars can be produced continuously by coupling this spraying and mixing step 1, kneading step 2 and second step 4, and sequentially sending and supplying the intermediates from a sulfuric acid spraying and mixing device to a hydrolysis reaction device.

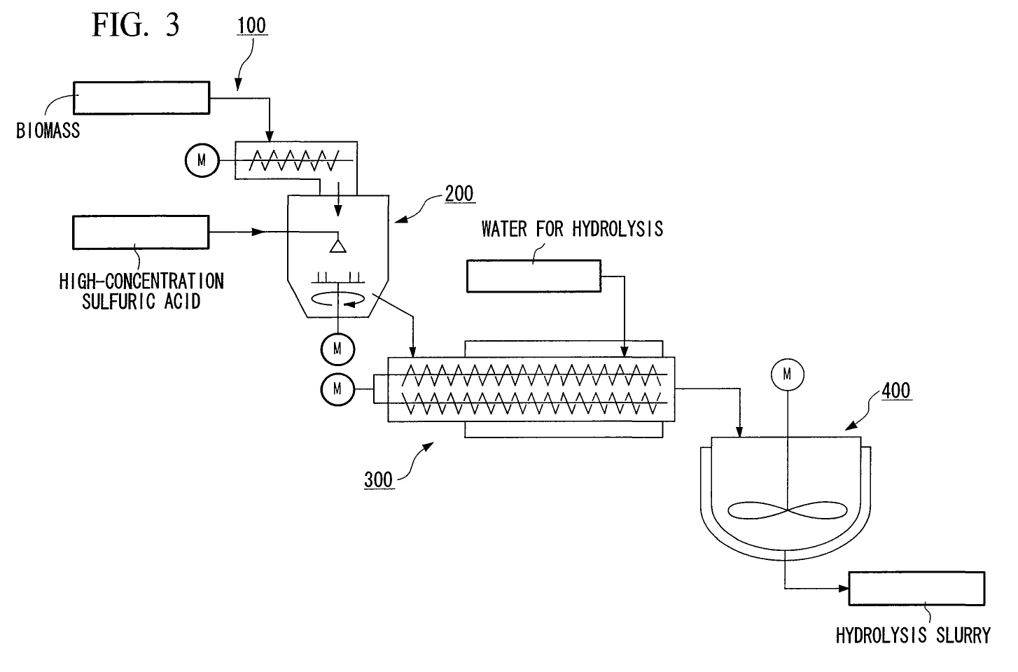

FIG. 3 shows a schematic drawing of a monosaccharide production device in which spraying and mixing, kneading, and hydrolysis are carried out continuously.

This monosaccharide production device is composed of a sulfuric acid spraying and mixing device 200, a continuous kneading device 300, and a hydrolysis reaction device 400. This device is composed such that intermediates are sequentially fed from sulfuric acid spraying and mixing device 200 to hydrolysis reaction device 400 continuously.

According to the monosaccharide production device shown in FIG. 3, a biomass is first sent by a raw material weighing and supply device 100 in the form of a screw feeder or table feeder and so forth to the sulfuric acid spraying and mixing device (biomass/sulfuric acid mixing device) 200.

In addition to being provided with a sprayer or shower for spraying highly concentrated sulfuric acid, this sulfuric acid spraying and mixing device 200 is also preferably provided with rotating blades for mixing the sulfuric acid and biomass. Within this sulfuric acid spraying and mixing device 200, in addition to being uniformly sprayed with highly concentrated sulfuric acid, the biomass is mixed as a result of being rotated by blades rotating at a comparatively high speed, resulting in the formation of a biomass sprayed and mixed with sulfuric acid. The concentration of sprayed sulfuric acid at this time is 65 to 85 (w/w) %, and preferably 70 to 75 (w/w) %, in the same manner as the first step 3.

Next, this biomass sprayed and mixed with sulfuric acid is sent to continuous kneading device 300 in the form of a kneader and so forth. This continuous kneading device 300 is used for the purpose of allowing the sulfuric acid to adequately permeate into the fine structure within the biomass that has uniformly been sprayed with sulfuric acid, as well as promoting the decrystallization and solubilization reactions of cellulose remaining in the biomass. Thus, this continuous kneading device 300 preferably has a mechanism which applies shear stress to the biomass sprayed and mixed with sulfuric acid. This biomass sprayed and mixed with sulfuric acid is heated to a temperature of 30 to 70° C., and preferably 40 to 55° C., in the same manner as the first step 3, and kneaded for 0.5 to 30 minutes while applying shear stress to obtain a kneaded product.

This kneaded product, which is in the form of a viscous gel as a result of kneading, is sent to plug flow type or CSTR type of hydrolysis reaction device 400 following addition of water or sulfuric acid for the hydrolysis reaction. This hydrolysis reaction device 400 preferably has a function capable of maintaining conditions which promote the hydrolysis reaction by uniformly dissolving the slurry in hot water. The conditions of this hydrolysis reaction consist of a sulfuric acid concentration of 20 to 60 (w/w)% and preferably 20 to 40 (w/w)%, a temperature of 40 to 100° C. and preferably 80 to 100° C., and a hydrolysis reaction time of 10 to 60 minutes.

Moreover, intermediates form in each device are sequentially sent to the following device from sulfuric acid spraying and mixing device 200 to hydrolysis reaction device 400 continuously. As a result of being able to sequentially send intermediates to the following device, the equipment scale and cost of this monosaccharide production device can be reduced.

In the present invention, since cellulose and hemicellulose can be simultaneously decrystallized and solubilized by setting the first step 3 to reaction conditions focused on the solubilization of cellulose, saccharification treatment of the subsequent second step 4 can be formed in a single treatment, making it possible to simplify the process as compared with the process of the “Arkenol process” of the prior art, which required the saccharification reaction in the form of a hydrolysis reaction to be carried out twice.

In addition, in the present invention, since saccharification treatment (hydrolysis reaction) is only carried out once, the amount of sulfuric acid used for the entire process can be reduced as compared with the “Arkenol process”.

The following provides a more detailed explanation of the present invention through examples thereof. The present invention is not limited in any way by these examples.

### EXAMPLE 1

(Batch Method)

700 g of pine chips (coniferous tree) having a moisture content of 9.1 (w/w) % and containing 414 g of holocellulose, and 1100 g of 71.5 (w/w)% sulfuric acid were charged into a mixing stirrer (Dalton) having a reactor volume of 10 liters followed by carrying out the pretreatment of the first step for 40 minutes at 50° C. Determination of the amount of sulfuric acid based on 100 (w/w)% conversion yielded a value of 786.5 g (1100 g×0.715), and calculation of the weight-based mixing ratio of sulfuric acid to biomass (absolute dry weight) yielded a value of 1.24.

Subsequently, hot water was charged into the reactor to dilute the sulfuric acid concentration to 30 (w/w) % followed by carrying out the saccharification treatment of the second step for 90 minutes at 85° C.

At this time, in order to investigate the degree of degradation of xylose present in the saccharification treatment liquid (second step treatment product), the concentration of xylose was measured every 10 minutes using a high-performance liquid chromatograph (HPLC) (Shimadzu).

The relationship between saccharification treatment time and xylose concentration wt % is shown in Table 1.

TABLE 1

| Reaction time (min) | Xylose concentration (wt %) |
|---|---|
| 10 | 0.30 |
| 20 | 0.38 |
| 30 | 0.43 |
| 40 | 0.45 |
| 50 | 0.48 |
| 60 | 0.51 |
| 70 | 0.51 |
| 80 | 0.52 |
| 90 | 0.50 |

Based on the results of Table 1, since the xylose concentration was nearly constant, a reduction in the amount of xylose during saccharification treatment attributable to degradation was not observed.

Next, the saccharification treatment liquid was cooled to about 40° C., and the filtration procedure of step 2A was carried out.

The monosaccharide concentration (wt %) of the resulting filtrate was measured using the aforementioned high-performance liquid chromatograph (HPLC). The amount of monosaccharide contained in the filtrate was calculated using the following equation based on that value and the total amount of liquid.
Amount of monosaccharide (g)=Total amount of liquid (weight)×monosaccharide concentration (wt %)

As a result, the amount of monosaccharides such as glucose, xylose and mannose contained in the filtrate (to simply be referred to as “monosaccharides”) was 249 g (after saccharification treatment).

Determination of the conversion rate of holocellulose to monosaccharides based on the weight of the holocellulose from the amount of monosaccharides yielded a value of 60.1 (w/w)%.

The sugar-acid separation of step 2B was carried out on the resulting filtrate using a simulated moving bed chromatographic separation device. At this time, the recovery rates for glucose and sulfuric acid were 99.0 (w/w)% and 97.2 (w/w)%, respectively.

The concentration of sulfuric acid in this effluent saccharification liquid (raffinate) was 1.0 (w/w)% by weight. The monosaccharification treatment of the third step was then carried out on this effluent saccharification liquid (raffinate) using an autoclave by holding at a temperature of 121° C. for 30 minutes.

Subsequently, the sugar liquid was collected and the monosaccharide concentration in the sugar liquid (wt %) was again measured using the aforementioned high-performance liquid chromatograph (HPLC) to calculate the amount of monosaccharides.

As a result, the amount of monosaccharides in the sugar liquid was 312 g. Determination of the conversion rate from holocellulose to monosaccharides based on the weight of holocellulose from the amount of monosaccharides yielded a value of 75.5 (w/w)%.

### EXAMPLE 2

(Batch Method)

2000 g of eucalyptus chips (coniferous tree) having a moisture content of 6.2 (w/w)% and containing 1296 g of holocellulose, and 3000 g of 75 (w/w)% sulfuric acid were charged in the same manner as Example 1 followed by carrying out pretreatment for 35 minutes at 54° C. Determination of the amount of sulfuric acid used based on 100 (w/w)% conversion yielded a value of 2250 g (3000 g×0.75), and calculation of the weight-based mixing ratio of sulfuric acid to biomass (absolute dry weight) yielded a value of 1.20.

Subsequently, hot water was added to dilute the sulfuric acid concentration to 33.5 (w/w)% followed by carrying out saccharification treatment for 60 minutes at 92° C.

Here, the concentration of xylose in the saccharification treatment liquid (second step treatment product) was measured in the same manner as Example 1.

The relationship between saccharification treatment time and xylose concentration (wt %) is shown in Table 1.

TABLE 2

| Reaction time (min) | Xylose concentration (wt %) |
|---|---|
| 10 | 1.28 |
| 20 | 1.70 |
| 30 | 1.99 |
| 40 | 2.10 |
| 50 | 2.07 |
| 60 | 2.01 |

Based on the results of Table 2, although a slight degree of degradation of xylose was observed, there were no large decreases in xylose concentration observed.

Next, the saccharification treatment liquid was cooled to about 40° C., and the filtration procedure was carried out.

The monosaccharide concentration (wt %) of the resulting filtrate was measured in the same manner as Example 1 to calculate the amount of monosaccharides in the filtrate.

As a result, the amount of monosaccharides contained in the filtrate was 848 g (after saccharification treatment).

Determination of the conversion rate of holocellulose to monosaccharides based on the weight of the holocellulose from the amount of monosaccharides yielded a value of 65.4 (w/w)%.

Sugar-acid separation was carried out in the same manner as Example 1. At this time, the recovery rates for glucose and sulfuric acid were 98.5 (w/w)% and 96.8 (w/w)%, respectively.

The concentration of sulfuric acid in this effluent saccharification liquid (raffinate) was 1.2 (w/w)%. Monosaccharification treatment was then carried out on this effluent saccharification liquid (raffinate) in the same manner as Example 1.

Subsequently, the sugar liquid was collected and the monosaccharide concentration in the sugar liquid (wt %) was measured in the same manner as Example 1 to calculate the amount of monosaccharides.

As a result, the amount of monosaccharides in the sugar liquid was 1040 g. Determination of the conversion rate from holocellulose to monosaccharides based on the weight of holocellulose from the amount of monosaccharides yielded a value of 80.2 (w/w)%.

### EXAMPLE 3

(Continuous Method)

Waste wood chips having a moisture content of 9 (w/w)% and holocellulose content based on absolute dry weight of 66.9 (w/w)%, and 75 (w/w)% sulfuric acid were charged into a continuous sulfuric acid spraying device (Funken Powdex, Flow Jet Mixer (trade name)) at supply rates of 37.6 kg/hr and 45.6 kg/hr, respectively, followed by uniformly mixing the waste wood chips and sulfuric acid.

At this time, conversion to the amount of holocellulose charged into the device yielded a value of 22.9 kg/hr. In addition, determination of the amount of sulfuric acid used based on 100 (w/w)% conversion yielded a value of 34.2 kg/hr (45.6 kg×0.75), and calculation of the weight-based mixing ratio of sulfuric acid to biomass (absolute dry weight) yielded a value of 1.0.

Next, the waste wood/sulfuric acid mixture discharged from the continuous sulfuric acid spraying device was supplied to a kneader-type continuous kneading device (Kurimoto Ltd., KRC Kneader (trade name)). The rotating speed of the kneader-type kneading device was adjusted so that the retention time of the waste wood/sulfuric acid mixture in the device was 10 minutes.

Hot water was supplied to the highly viscous kneaded product discharged from the kneader-type kneading device so that the sulfuric acid concentration was 30 (w/w)% to form a slurry. This slurry was then sent to a hydrolysis reaction device, and after being discharged from the hydrolysis reactor device having a reaction temperature of 90° C. following a retention time of 30 minutes, the hydrolysis reaction product was cooled followed by carrying out the filtration procedure.

The monosaccharide concentration (wt %) of the resulting filtrate was measured in the same manner as Example 1 to calculate the amount of monosaccharides in the filtrate.

As a result, the amount of monosaccharides contained in the filtrate per hour was 14.4 kg.

Determination of the conversion rate of holocellulose to monosaccharides based on the weight of the holocellulose from the amount of monosaccharides yielded a value of 63.1 (w/w)%.

Sugar-acid separation was carried out in the same manner as Example 1 using the amount of filtrate obtained from 1 hour of operation. At this time, the recovery rates for glucose and sulfuric acid were 98.5 (w/w)% and 97.0 (w/w)%, respectively.

The concentration of sulfuric acid in this effluent saccharification liquid (raffinate) was 1.1 (w/w)%. Monosaccharification treatment was then carried out on this effluent saccharification liquid (raffinate) in the same manner as Example 1.

Subsequently, the sugar liquid was collected and the monosaccharide concentration in the sugar liquid (wt %) was measured in the same manner as Example 1 to calculate the amount of monosaccharides.

As a result, the amount of monosaccharides in the sugar liquid was 17.7 kg. Determination of the conversion rate from holocellulose to monosaccharides based on the weight of holocellulose from the amount of monosaccharides yielded a value of 77.3 (w/w)%.

### COMPARATIVE EXAMPLE 1

(Arkenol Process)

1.0 kg of cedar chips (coniferous tree) having a moisture content of 6.7 (w/w)% and containing 0.634 g of holocellulose, and 1.1 kg of 72 (w/w)% sulfuric acid were charged into a container in the same manner as Example 1 followed by carrying out decrystallization treatment for 45 minutes at 28° C.

Subsequently, hot water was added thereto to dilute the sulfuric acid concentration to 30 (w/w) %, followed by carrying out the first stage hydrolysis reaction treatment for 90 minutes at 95° C.

Next, this treatment liquid was cooled to about 40° C. followed by the first stage filtration procedure.

The monosaccharide concentration (wt %) of the resulting first stage filtrate was measured in the same manner as Example 1 to calculate the amount of monosaccharides.

As a result, the amount of monosaccharides in the first stage filtrate was 0.310 kg.

Determination of the conversion rate of holocellulose to monosaccharides based on the weight of the holocellulose from the amount of monosaccharides in the first stage hydrolysis reaction yielded a value of 48.8 (w/w)%.

1.45 kg of 30 (w/w)% by weight sulfuric acid were added to 2.0 kg of the solid (filter cake) obtained from the first stage filtration procedure followed by carrying out the second stage hydrolysis reaction treatment for 30 minutes at 95° C.

Determination of the amount of sulfuric acid used based on 100 (w/w)% conversion there from yielded a value of 1.23 kg (1.1 kg×0.72+1.45×0.3), and calculation of the weight-based mixing ratio of sulfuric acid to biomass (absolute dry weight) yielded a value of 1.32.

Next, this treatment liquid was cooled to about 40° C. followed by carrying out the second stage filtration procedure.

The monosaccharide concentration (wt %) of the resulting second stage filtrate was measured in the same manner as Example 1 to calculate the amount of monosaccharides.

As a result, the amount of monosaccharides in the second stage filtrate was 0.196 kg. This is the value after the second stage hydrolysis reaction that includes monosaccharides adhered to the solid following the first stage hydrolysis reaction. Thus, it is necessary to subtract the amount of monosaccharides formed in the first stage hydrolysis reaction which adhered to the solid used as the raw material in the second stage hydrolysis reaction.

The amount of monosaccharides after subtracting in this manner was 0.047 kg (value of the amount of monosaccharides only for after the second stage hydrolysis reaction). In addition, determination of the conversion rate from holocellulose to monosaccharides based on the weight of holocellulose from the amount of monosaccharides in the second stage hydrolysis reaction yielded a value of 7.2 (w/w)%.

Determination of the final conversion rate from holocellulose to monosaccharides according to the “Arkenol process” (two-stage hydrolysis method) using cedar for the raw material from the amounts of monosaccharides obtained in the first stage hydrolysis reaction and second stage hydrolysis reaction yielded a value of 56.0 (w/w)%.

When comparing Example 1 to 3 with Comparative Example 3, in contrast to the final conversion rate of holocellulose to monosaccharides based on the weight of holocellulose being less than 60 (w/w)% in Comparative Example 1, those values for Examples 1 to 3 demonstrate high conversion rates of 75 (w/w)% or more.

On the basis of these results, reductions in concentration caused by degradation of xylose were confirmed to not have occurred during saccharification treatment (hydrolysis reaction). The method for producing monosaccharides of the present invention was also confirmed to demonstrate a high monosaccharide conversion rate even though saccharification treatment was carried out in a single treatment.

### INDUSTRIAL APPLICABILITY

The present invention can be applied to fields involving the discharge of fiber-based biomass (including the construction and food product fields), fields involving alcohol production, fields involving the production of alcohol-mixed fuels, and fields using glucose as a fermentation raw material (carbon source) (including fields involving the production of polylactic acid and amino acids).

FIG. 1

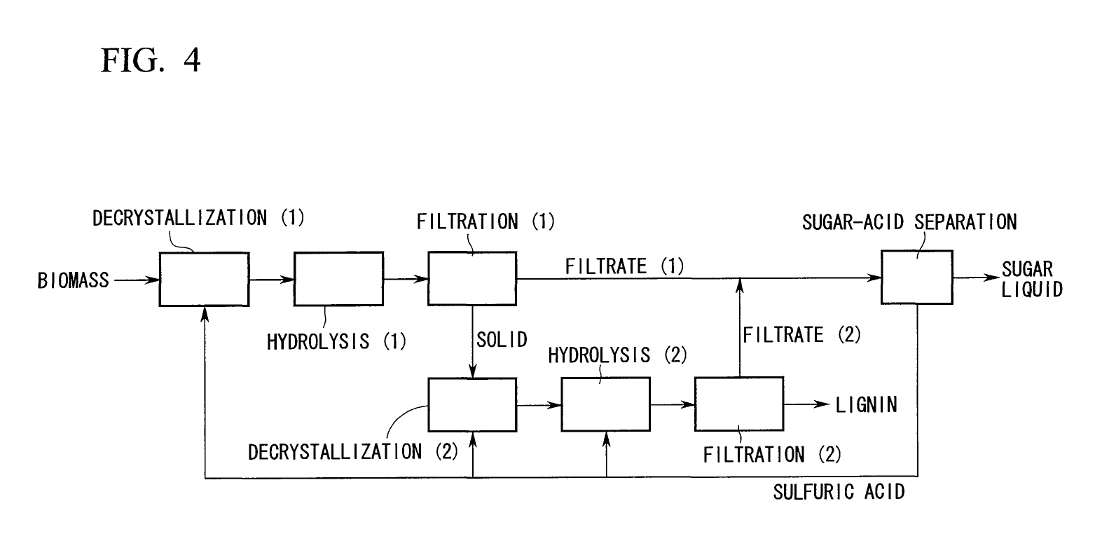

Biomass
High-concentration sulfuric acid
1 Spraying/mixing
2 Kneading
3 Pretreatment
4 Saccharification treatment
Washing filtrate
Washing water
5 Filtration
Lignin
Filtrate
6 Sugar-acid separation
Saccharification liquid sulfuric acid
7 Monosaccharification treatment
Sugar liquid
8 Sulfuric acid recovery/concentration
High-concentration Sulfuric acid
   FIG. 2
Biomass
High-concentration sulfuric acid
1 Spraying/mixing
2 Kneading
3 Pretreatment
4 Saccharification treatment
Washing filtrate
Washing water
Low-concentration sulfuric acid
5 Filtration
Lignin
Filtrate
6 Sugar-acid separation
Saccharification liquid
Low-concentration sulfuric acid
High-concentration sulfuric acid
7 Monosaccharification treatment
Sugar liquid
8 Sulfuric acid concentration
Low-concentration sulfuric acid
High-concentration sulfuric acid
   FIG. 3
Biomass
High-concentration sulfuric acid
Water for hydrolysis
Hydrolysis slurry
   FIG. 4
Biomass
Decrystallization (1)
Hydrolysis (1)
Filtration (1)
Solid
Filtrate (1)
Filtrate (2)
Sugar-acid separation
Sugar liquid
Decrystallization (2)
Hydrolysis (2)
Filtration (2)
Lignin
Sulfuric acid

1. A method for producing monosaccharides from biomass comprising: a first step in which a biomass is pretreated in 65 to 85 (w/w)% sulfuric acid at a temperature of 30 to 70° C., and a second step in which the first step treatment product pretreated in the first step is subjected to saccharification treatment in 20 to 60 (w/w)% sulfuric acid at a temperature of 40 to 100° C.

2. The method for producing monosaccharides according to claim 1 having a third step in which the second step treatment product resulting from saccharification treatment in the second step is subjected to monosaccharification treatment in 0.5 to 5 (w/w)% sulfuric acid at a temperature of 110 to 150° C.

3. The method for producing monosaccharides according to claim 1 further having a step 2A, in which the treatment product of the second step resulting from saccharification treatment in the second step is subjected to filtration, and a step 2B, in which the filtrate following step 2A is separated into sugar and acid.

4. The method for producing monosaccharides according to claim 1 having a step in which the sulfuric acid is sprayed onto and mixed with the biomass followed by kneading.

5. The method for producing monosaccharides according to claim 1, wherein the weight-based mixing ratio of the sulfuric acid to biomass is 0.3 to 5.0.

6. The method for producing monosaccharides according to claim 3, wherein a washing filtrate, obtained by washing the solid after step 2A, is used.

7. The method for producing monosaccharides according to claim 3, wherein a simulated moving bed chromatographic separation device is used for the separation into sugar and acid in step 2B.

8. The method for producing monosaccharides according to claim 3, wherein low-concentration sulfuric acid after step 2B is used for the sulfuric acid of the second step.

9. The method for producing monosaccharides according to claim 1, wherein the biomass is a cellulose-based biomass.

10. A monosaccharide production device provided with: a sulfuric acid spraying and mixing device, which sprays 65 to 85 (w/w)% sulfuric acid onto a biomass and mixes the sulfuric acid and biomass by rotating to obtain a sulfuric acid-sprayed/mixed biomass, a continuous kneading device which kneads the sulfuric acid-sprayed/mixed biomass from the sulfuric acid spraying and mixing device by applying shear force to obtain a kneaded product, and a hydrolysis reaction device which adds water or low-concentration sulfuric acid to the first step treatment product in the form of the kneaded product from the continuous kneading device to dilute the sulfuric acid concentration to 20 to 60 (w/w)% followed by treatment at a temperature of 40 to 100° C.; wherein, sequential intermediate products are continuously sent from the sulfuric acid-spraying/mixing device to the hydrolysis reaction device.
